(19) United States
(12) Patent Application Publication
(10) Pub. No.: US 2023/0265077 A1
(43) Pub. Date: Aug. 24, 2023

(54) Photoactive Compound, Photoactive Protein-Immobilizing Gel And Use

(71) Applicant: Water Bear Health Technology (Nantong) Co., LTD., Nantong City (CN)

(72) Inventors: Xianting Ding, Shanghai (CN); Haiyang Xie, Shanghai (CN); Ting Zhang, Shanghai (CN); Shanhe Li, Shanghai (CN)

(21) Appl. No.: 17/677,565

(22) Filed: Feb. 22, 2022

(51) Int. Cl. C07D 403/04; C07K 1/26; C07D 409/04; C07D 405/04; C07D 257/04

(52) U.S. Cl. CPC C07D 403/04; C07K 1/26; C07D 409/04; C07D 405/04; C07D 257/04

(57) ABSTRACT

Provided are a photoactive compound having a chemical structure of formula (I), a photoactive protein-immobilizing gel and use. The photoactive protein-immobilizing gel, in which the photoactive compound is contained or immobilized, can crosslink with a protein under the action of UV light, thereby achieving separation and/or immobilization of the protein.

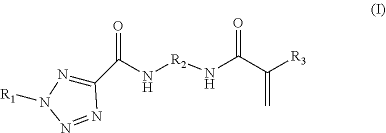

### FIELD OF THE INVENTION

The present invention relates to the technical field of chemical biology and, in particular, to a photoactive compound, a photoactive protein-immobilizing gel and use.

### DESCRIPTION OF THE PRIOR ART

In the field of tumor research, tumor cells themselves are highly heterogeneous, and targeted therapy for cancer requires quantitative and highly specific detection of targeted proteins at the level of single-cell resolution. The study of cellular heterogeneity can provide abundant critical genetic expression information for individualized therapies and lay the foundations for the determination of treatment plan and for the selection and development of targeted drugs. In the field of stem cell research, the study of gene expression differences between different cells helps to deepen the understanding of mechanisms of genetic development and gene expression regulation in different tissues and cells. Proteins are the material basis of life, organic macromolecules, the basic organic matter that builds cells, and the main undertaker of life activities. Epigenetic modifications such as acetylation, ubiquitination and phosphorylation play a crucial role in the regulation of proteins' functions. Genomic and transcriptomic research cannot satisfy the desire of human being to explore heterogeneity present in the emergence and evolution of diseases, and this explains the emergence of single-cell proteomics.

At present, microfluidic chip technology, fluorescence flow cytometry, mass cytometry, imaging mass cytometry, single-cell secretory protein detection technology, single-cell western blot and other high-throughput, high-sensitivity, high-resolution analytic techniques are powerful tools available for single-cell omic studies. Western blot is a protein assay method commonly used in cell and molecular biology and immunogenetics. It specifically involves separating proteins from a sample by means of gel electrophoresis, transferring the proteins to a solid carrier (e.g., nitrocellulose or PVDF) and detecting the proteins in the sample with an antibody specific for the targeted protein. Since the proteins are bound with the antibody after being separated by electrophoresis, less impact of cross-reactivity of the antibody is achieved. Therefore, on-target and off-target signals can be clearly distinguished even in complex samples such as cell lysates.

However, the result of the traditional western blot is based upon an average protein expression level of a large number of cell samples, which masks the specificity and diversity of single cells' protein expression. Since antigen binds to antibody with certain degree of specificity, protein immunoassay methods relying only on antigen-antibody binding for the identification of targeted protein molecules, such as microfluidic technology, fluorescence flow cytometry, mass cytometry, imaging mass cytometry, isoplex's single-cell secretion detection technique, etc., are of low specificity and highly likely to produce false positive results. Moreover, due to a limited types of available specific probes, these methods are limited in application. Further, regarding the detection of cell surface protein, transmembrane protein, secreted protein and intracellular or even intranuclear protein, it is difficult in terms of operation for flow cytometry to directly detect intracellular and intranuclear proteins, and single-cell secretory protein detection technology is only applied in detecting secreted protein but not cell surface protein, transmembrane protein, intracellular protein and intranuclear proteins.

Combining the molecular sieving ability of sodium dodecyl sulfate (SDS)-polyacrylamide gels (PAGE) and the specificity of western blot, single-cell western blot not only ensures detection specificity through double verification consisting of protein molecular weight determination and antigen-antibody recognition, but is also applied in detecting cell surface protein, transmembrane protein, intracellular protein, intranuclear protein and distinguishable epigenetically modified protein. However, N-(3-[(4-benzoylphenyl)formamido]propyl)methacrylamide-based photoactive gel is of low photoactive immobilization efficiency, and its lower detection limit is 27,000 protein molecules from a single cell, which makes it difficult for use in the detection of low-abundance proteins. Additionally, the photoactive gel produces higher background fluorescence, and the sensitivity of the method is low. Further, its excitation wavelength is relatively short (UVB, 280-320 nm), and may deactivate protein antigenic site, which would produce false negative result. Furthermore, it is difficult for use in single-cell protein analysis of rare cell populations such as circulating tumor cells (1-10 cells/ml blood).

### SUMMARY OF THE INVENTION

The present invention relates to a photoactive compound, which, as a photoactive ingredient, can be cross linked with a protein under the action of ultraviolet (UV) light, thereby immobilizing and/or separating the protein.

In one embodiment, the present invention provides a photoactive compound having a structure of formula (I):

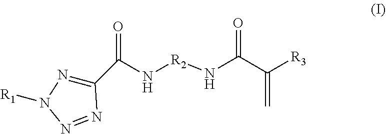

wherein: R₁ is selected from nitro, cyano, halogen, haloalkyl, —ORₐ, —S(═O)_{q}—Rₐ, —C(═Y)—Rₐ, —NRₐR_{b}, substituted or unsubstituted heterocyclyl and substituted or unsubstituted aryl, wherein Rₐ and R_{b} of each occurrence in R₁ are same or different and independently selected from hydrogen, nitro, hydroxyl, cyano, halogen, substituted or unsubstituted alkyl and substituted or unsubstituted cycloalkyl, and R₁ is preferred to be an electronegative group which not only allows increased chemical stability of the tetrazolyl functional group that it is joined to but also promotes a reaction of the tetrazolyl ring with protein molecules under UV light; R₂ is selected from —NH—, —O—, —S—, —C(═Y)—, —C(═Y)—CRₐR_{b}—, —CRₐR_{b}—C(═Y)—CRₐR_{b}—, —Y—CRₐR_{b}—, —CRₐR_{b}—Y—CRₐR_{b}—, —C(═Y)—NRₐ—, —NRₐ—C(═Y)—NRₐ—, —S(═O)_{q}—NRₐ—, —NRₐ—S(═O)_{q}—NRₐ—, —S(═O)_{q}—CRₐR_{b}—, —CRₐR_{b}—S(═O)_{q}—CRₐR_{b}—, substituted or unsubstituted alkyl, substituted or unsubstituted alkenyl, substituted or unsubstituted alkynyl, substituted or unsubstituted cycloalkyl, substituted or unsubstituted heterocyclyl and substituted or unsubstituted aryl, wherein Rₐ and R_{b} of each occurrence in R₂ are same or different and independently selected from hydrogen, nitro, hydroxyl, cyano, halogen, substituted or unsubstituted C₁₋₆ alkyl, substituted or unsubstituted C₂₋₆ alkenyl, substituted or unsubstituted C₂₋₆ alkynyl, substituted or substituted C₃₋₆ cycloalkyl and substituted or unsubstituted C₃₋₆ cycloalkenyl; R₃ is selected from hydrogen, —NRₐR_{b}, —C(═Y)—Rₐ, —CRₐR_{b}—C(═Y)—Rₐ, —CRₐR_{b}—Y—Rₐ, —C(═Y)—NRₐR_{b}, —NRₐ—C(═Y)—NRₐR_{b}, —S(═O)_{q}—NRₐR_{b}, —NRₐ—S(═O)_{q}—NRₐR_{b}, —S(═O)_{q}—Rₐ, —CRₐR_{b}—S(═O)_{q}—Rₐ, substituted or unsubstituted alkyl, substituted or unsubstituted cycloalkyl, substituted or unsubstituted heterocyclyl and substituted or unsubstituted aryl, wherein Rₐ and R_{b} of each occurrence in R₃ are the same or different and independently selected from hydrogen, nitro, hydroxyl, cyano, halogen, substituted or unsubstituted alkyl and substituted or unsubstituted cycloalkyl; Y of each occurrence is independently selected from 0, S and NRₐ; and q of each occurrence independently represents 0, 1 or 2.

More preferably, in the compound of formula (I), R₁ is selected from any of the following groups (where each wavy line

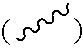

represents a position where the group is connected with the rest of the compound):

—NO₂, CN, —Cl, —F, —Br, —CH₂Cl, —CHCl₂, —CCl₃, —CH₂F, —CHF₂, —CF₃, —OCH₃, —OCH₂CH₃, —COOH, —C(═O)—CH₃, —C(═O)—CH₂CH₃, —C(═O)—CH₂CN, —C(═O)—CH₂NO₂, —SCH₃, —S(═O)₂—OH, —S(═O)₂—CH₃, —S(═O)₂—CH₂CN, —S(═O)₂—CH₂CH₂NO₂, —NCH₃CH₃, —NHCH₃, —NHCH₂CN, —NHCH₂NO₂,

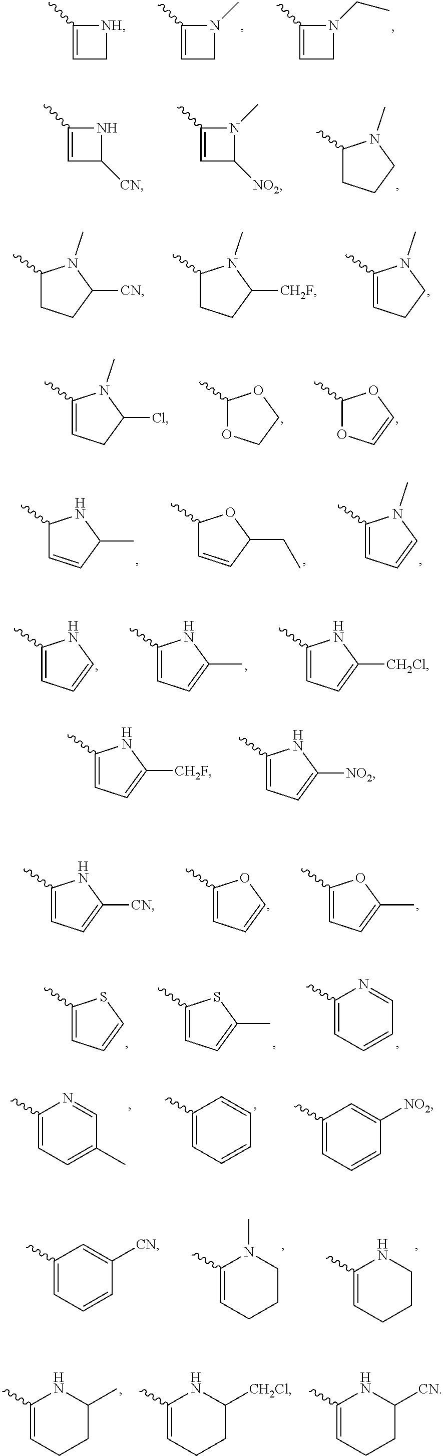

More preferably, in the compound of formula (I), R₂ is selected from any of the following groups:

—C(═O)—, —C(═O)—CH₂—, —CH₂—C(═O)—CH₂—, —CH₂—, —CH₂—CH₂—, —CH(CH₃)—, —CH₂—CH₂—CH₂—, —CH₂—CH(CH₃)—CH₂—, —CH═CH—, —CH═C(CH₃)—, —C(CH₃)═C(CH₃)—, —CH₂—CH═CH—CH₂—, —CH₂—NH—CH₂—, —NH—CH₂—, —S(═O)₂—CH₂—, —CH₂—S(═O)₂—CH₂—, —CH(OH)—, —CH₂CH(OH)CH₂—, —CH(CN)—, —CH₂CH(CN)CH₂—, —CH(NO₂)—, —CH₂CH(NO₂)CH₂—, —CH₂—O—CH₂—, —CH₂—S—CH₂—.

More preferably, in the compound of formula (I), R₃ is selected from any of the following groups:

—NH₂, —NHCH₃, —CH₃, —CH₂CH₃, —CH₂CH₂CH₃, —CH(OH)CH₃, —C(═O)—CH₃, —CH₂—C(═O)—CH₃, —C(═O)—NH₂, —CH₂—O—CH₃, —CH₂—S—CH₃, —NH—S(═O)₂—NH—CH₃, —S(═O)₂—CH₂CH₃, —CH₂—S(═O)₂—CH₃,

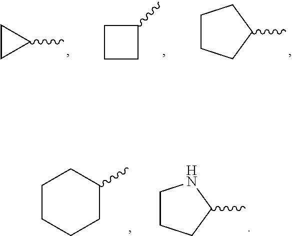

More preferably, in the compound of formula (I), R₁ is substituted or unsubstituted heterocyclyl or substituted or unsubstituted aryl.

More preferably, in the compound of formula (I), R₂ is substituted or unsubstituted alkyl.

More preferably, in the compound of formula (I), R₃ is substituted or unsubstituted alkyl.

More preferably, in the compound of formula (I), R₂ is substituted or unsubstituted C₁₋₆ alkyl and R₃ is substituted or unsubstituted C₁₋₆ alkyl.

More preferably, in the compound of formula (I), R₁ is selected from any of the following groups:

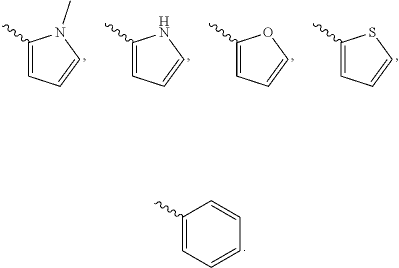

More preferably, in the compound of formula (I), R₂ is unsubstituted C₁₋₆ alkyl.

More preferably, in the compound of formula (I), R₃ is unsubstituted C₁₋₆ alkyl.

The representative compounds of formula (I) in the present invention include the following named compounds, but the invention should not be construed as being limited to these compounds:

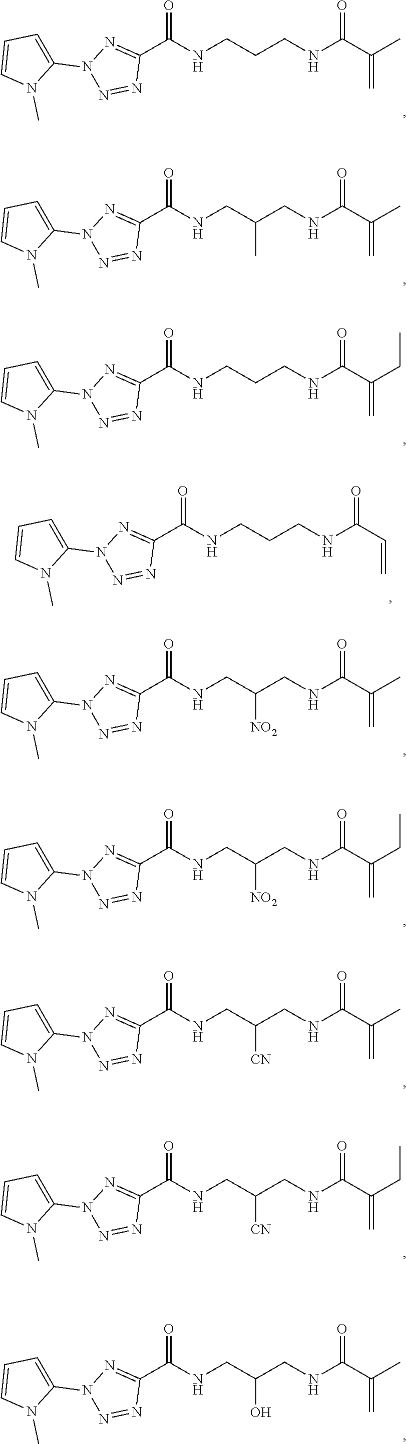

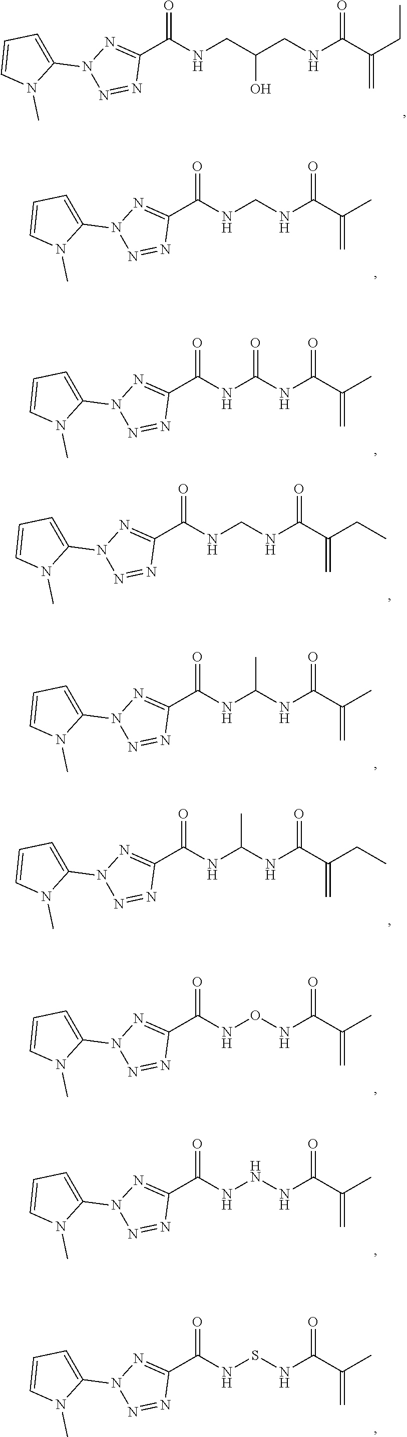

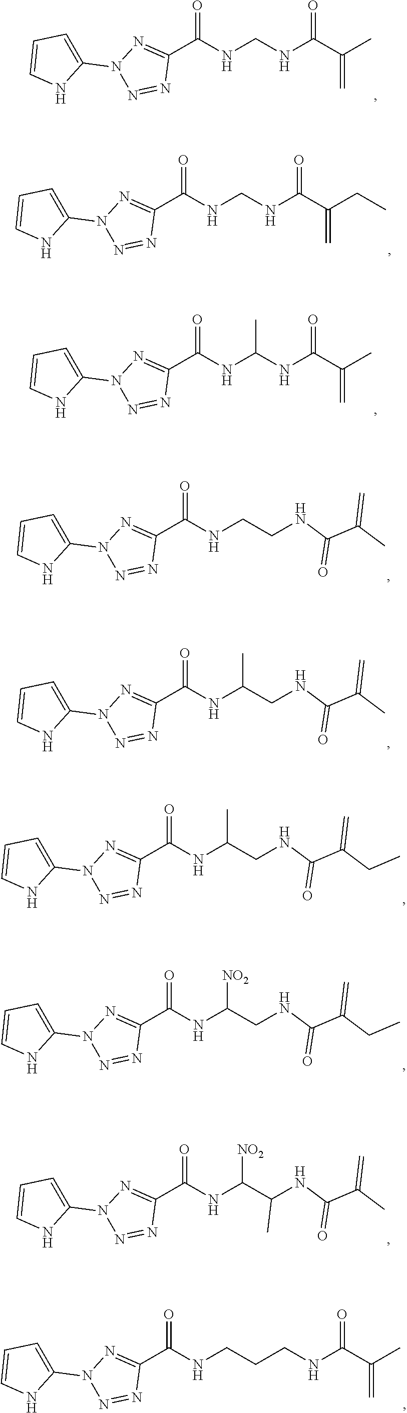

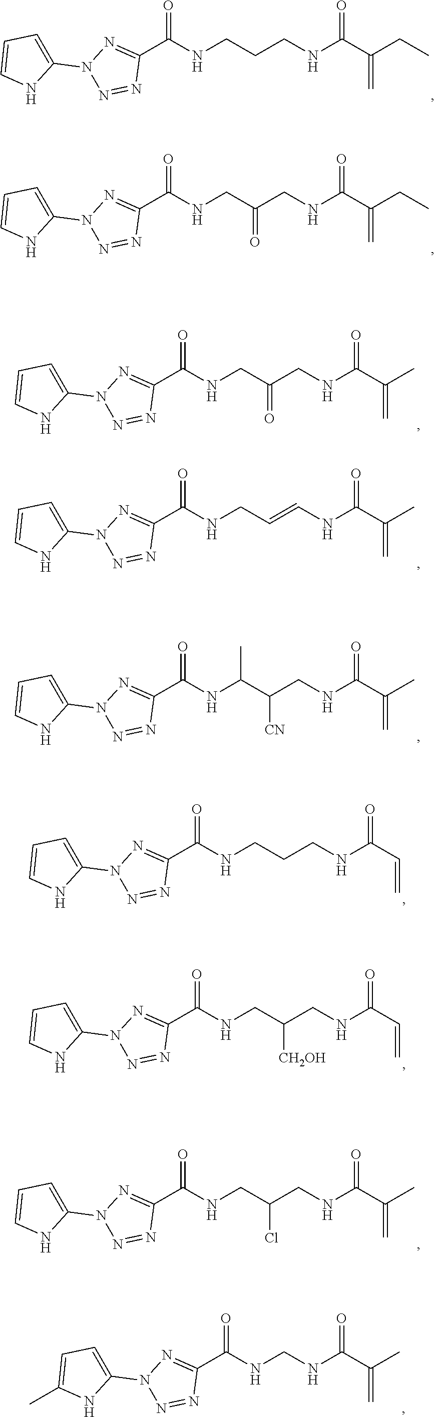

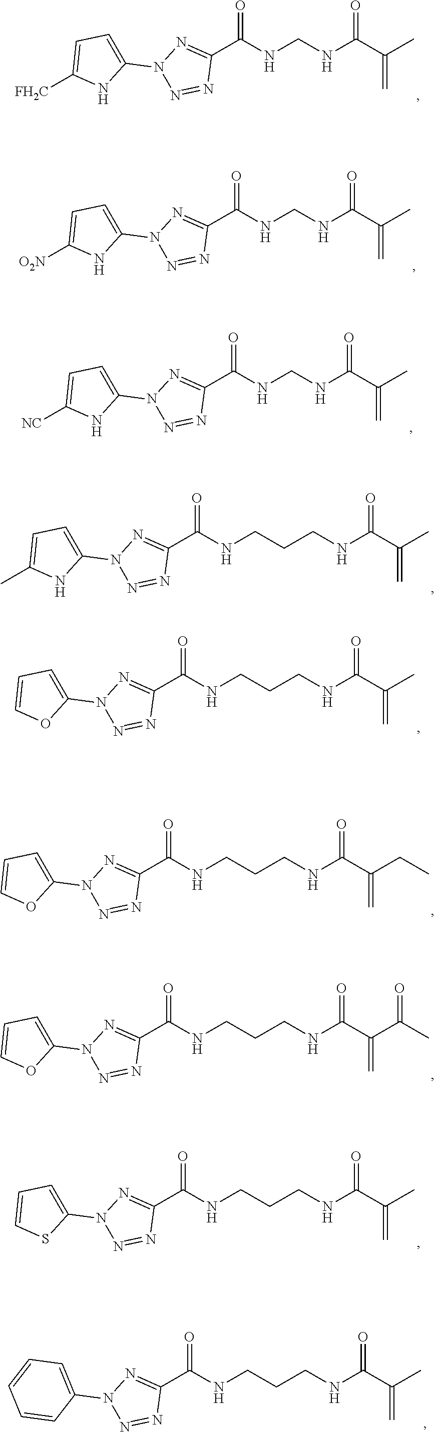

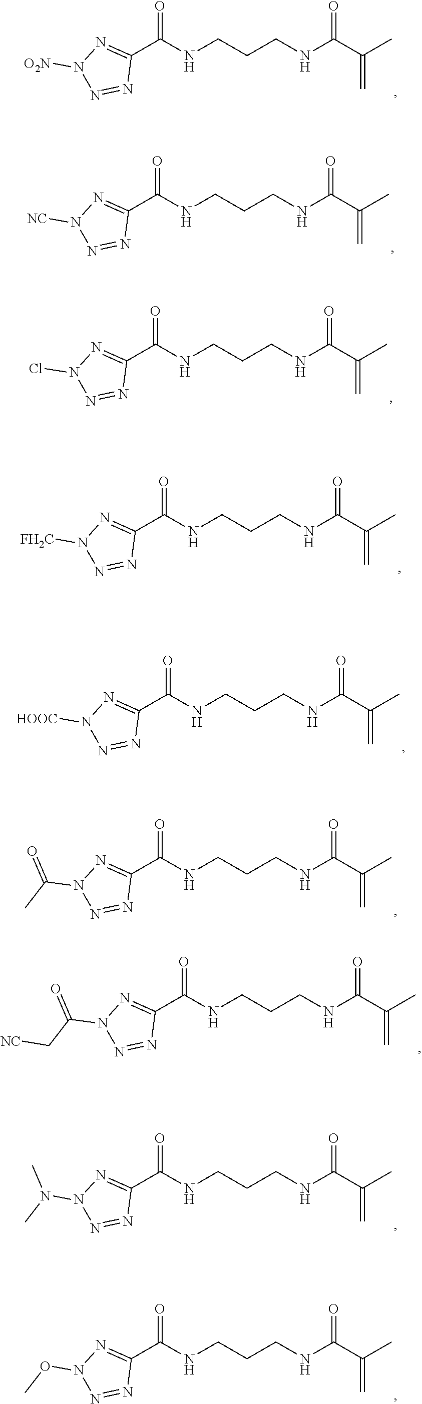

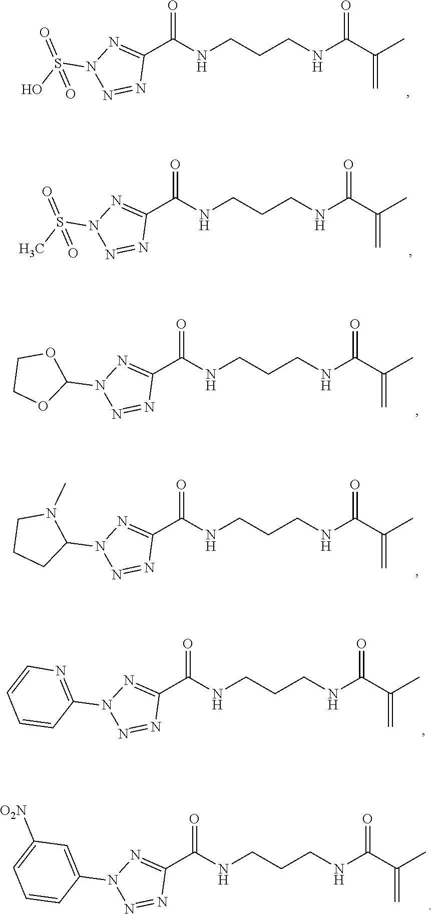

In another embodiment of the present invention, it provides a photoactive protein-immobilizing gel, in which a photoactive compound having a chemical structure of formula (I) is contained or immobilized.

More specifically, the gel may undergo a polymerization reaction with the rightmost double bond of the photoactive compound having the structure of formula (I) (i.e., the double bond directly connected to R₃), resulting in the photoactive compound directly connecting with the gel, i.e., the photoactive compound is immobilized on the gel.

More preferably, the gel is a polyacrylamide hydrogel.

In another embodiment of the present invention, it provides a use of a photoactive compound having a chemical structure of formula (I) for separating a protein.

More specifically, the tetrazolyl ring based compound of the present invention is a photoactive ingredient which can be used in technologies with separation function, such as SDS-PAGE electrophoresis, isoelectric focusing electrophoresis, non-denaturing PAGE, two-dimensional electrophoresis, mass-to-charge ratio separation and affinity separation.

In another embodiment of the present invention, it provides a use of the photoactive protein-immobilizing gel as defined above for separating, immobilizing and/or detecting a protein.

More specifically, the present invention can integrate protein separation function and protein immobilization function, and can be used in microfluidic western blot, single-cell western blot, capillary western blot and other technologies. The photoactive compound of the present invention has a tetrazolyl ring as a photoactive group, and the photoactive protein-immobilizing gel contains the photoactive compound as a photoactive ingredient which can react with a functional group (—COOH) in a protein when being excited by UV light, thereby crosslinking and immobilizing the protein within the gel. Thus, it can be applied in subsequent in-situ specific quantitative or semi-quantitative detection of a targeted protein within the gel.

In the photoactive compound provided in the present invention, the tetrazolyl ring serves as a photoactive group, and an electron-withdrawing group thereof is modified to tune wavelength and energy of desired excitation light. Under the action of UV excitation, the tetrazolyl ring will be split up and then crosslink with a protein, thereby immobilizing the protein. Therefore, the photoactive compound can be used as a photoactive ingredient to prepare a photoactive protein-immobilizing gel for use in research involving single-cell western blot.

When the photoactive protein-immobilizing gel of the present invention is employed in a western blot assay, antibody probe for detection may be labeled with fluorescence, enzyme, colloidal gold, super-paramagnetic microspheres, etc., and detection reaction may be with reagent other than antibody, such as aptamer, nanobody, agglutinin, etc. The assay can be used to detect single-cell or multiple-cells, extracted protein, solution of a purified protein, or the like. It can be also used for simultaneous detection of one or more protein analytes.

The photoactive protein-immobilizing gel prepared with the photoactive compound of the present invention allows protein to be in-situ immobilized therein stably, and the immobilization is stronger and more efficient without easy elution and loss of the protein, which ensures the sensitivity of subsequent detection. Moreover, it polymerizes with functional group in protein at a faster rate, which greatly promotes protein immobilization efficiency and avoids protein diffusion that may lead to impaired sensitivity due to a slow polymerization rate.

In addition, during immobilization of protein in the gel, the photoactive group is excited by UV light in a shorter time, which allows faster protein immobilization. UV light suitable for the excitation has longer wavelengths in a narrower band and lower energy, and can reduce UV-induced background autofluorescence from the gel itself, avoid deactivation of an antigenic site of the protein and save detection time and cost.

The photoactive protein-immobilizing gel of the present invention can non-selectively immobilize all proteins, including those of different molecular weights, different acidity or basicity, different isoelectric points, different three-dimensional structures, different locations (e.g., surface proteins, transmembrane proteins, intracellular proteins and intranuclear proteins), etc. This avoids missed or false detection of a particular protein due to the method itself. The non-selectivity makes it also usable in other western blot assays (e.g., DNA-protein or RNA-protein).

Further, in experimental systems of single-cell western blot technology, the photoactive protein-immobilizing gel of the present invention does not react with other molecules, such as water, SDS, Triton X, sodium deoxycholate, etc., which avoids decreases in protein immobilization efficiency resulting from the occupation of immobilization sites by those molecules, as well as background fluorescence caused thereby. In addition, it does not affect antigen-antibody binding, receptor-ligand binding, enzymatic activity, aptamer binding, or the like, which ensures subsequent successful and efficient establishment of a detection system.

Finally, the photoactive protein-immobilizing gel is allowed to be used at a very low concentration for protein immobilization, which does not affect the molecular sieving of SDS-PAGE, and allows the method to retain its own specificity and separation resolution.

### Description and Definition of Terminology

As used herein, unless otherwise indicated, the following definitions shall apply. The substituents listed in the definitions are exemplary and shall not be construed as limiting substituents defined elsewhere in the specification.

The term “alkyl” refers to a straight- or branched-chain hydrocarbon group consisting solely of carbon and hydrogen atoms, containing no unsaturation, having 1 to 8 carbon atoms, and connecting with the rest of the molecule by a single bond, such as methyl, ethyl, n-propyl, 1-methylethyl (isopropyl), n-butyl, n-pentyl and 1,1-dimethylethyl (tert-butyl).

The term “substituted or unsubstituted C₁₋₆ alkyl” refers to an alkyl group as defined above with up to 6 carbon atoms.

The term “alkenyl” refers to an aliphatic hydrocarbon group containing a carbon-carbon double bond and which may have a straight or branched chain with about 2 to about 10 carbon atoms, such as ethenyl, 1-propenyl, 2-propenyl (allyl), iso-propenyl, 2-methyl-1-propenyl, 1-butenyl, and 2-butenyl.

The term “substituted or unsubstituted C₂₋₆ alkenyl” refers to an alkenyl group as defined above with up to 6 carbon atoms.

The term “alkynyl” refers to a straight- or branched-chain hydrocarbon group with at least one carbon-carbon triple bond and 2 to 12 carbon atoms (currently, groups with about 2 to 10 carbon atoms being preferred), such as ethynyl, propynyl and butynyl.

The term “substituted or unsubstituted C₂₋₆ alkynyl” refers to an alkynyl group as defined above with up to 6 carbon atoms.

The term “cycloalkyl” refers to a non-aromatic monocyclic or polycyclic ring system of about 3 to 12 carbon atoms, such as cyclopropyl, cyclobutyl, cyclopentyl and cyclohexyl. Examples of polycyclic cycloalkyl groups include perhydronapththyl, adamantyl and norbornyl groups, bridged cyclic groups and spirobicyclic groups such as spiro(4,4)non-2-yl.

The term “substituted or unsubstituted C₃₋₆ cycloalkyl” refers to a cycloalkyl group as defined above with up to 6 carbon atoms.

The term “cycloalkenyl” refers to a cyclic ring-containing group containing about 3 to 8 carbon atoms and with at least one carbon-carbon double bond, such as cyclopropenyl, cyclobutenyl and cyclopentenyl.

The term “substituted or unsubstituted C₃₋₆ cycloalkenyl” refers to a cycloalkenyl group as defined above with up to 6 carbon atoms.

The term “aryl” refers to a monocarbocyclic aromatic group, or a fused or non-fused polycarbocyclic aromatic group, which has 6 to 14 carbon atoms. In the polycarbocyclic case, there may be only one aromatic carbon ring. Aryl groups also include those fused with heterocyclic rings. Examples of aryl include phenyl, biphenyl and naphthyl.

The term “heterocyclyl” refers to a 3- to 15-membered ring group consisting of carbon atoms and at least one heteroatom selected from nitrogen, oxygen and sulfur.

The heterocyclyl group may be a monocyclic, bicyclic, tricyclic or tetracyclic ring system, which may include fused, bridged or spiro ring systems, and the nitrogen, carbon, oxygen or sulfur atoms in the heterocyclyl group may be optionally oxidized to various oxidation states. In addition, the nitrogen atom may be optionally quaternized. The heterocyclyl group may be attached to the main structure at any heteroatom or carbon atom that results in the generation of a stable structure. Heterocyclyl may be aromatic or non-aromatic. Examples of heterocyclyl include azetidinyl, pyrrolidinyl, pyrrolinyl, tetrahydrofuranyl, dihydrofuranyl, piperazinyl, piperidinyl, morpholinyl, thiomorpholinyl, tetrahydropyranyl, tetrahydrothienyl, etc.

Unless otherwise specified, the term “substituted” refers to substitution with any one or any combination of the following substituents which may be the same or different, the one or more substituents selected from, for example, the following groups: hydrogen, hydroxyl, halogen, carboxyl, cyano, nitro, oxo (═O), thio (═S), substituted or unsubstituted alkyl, substituted or unsubstituted alkoxyl, substituted or unsubstituted alkenyl, substituted or unsubstituted alkynyl, substituted or unsubstituted cycloalkyl, substituted or unsubstituted cycloalkenyl, substituted or unsubstituted heterocyclyl, substituted or unsubstituted aryl, —COOR′, —C(O)R′, —C(S)R′, —C(O)NR′R″, —C(O)ONR′R″, —NR′R″, —NR′CONR′R″, —N(R′)SOR″, —N(R′)SO₂R″, —(═N—N(R′)R″), —NR′C(O)OR″, —NR′R″, —NR′C(O)R″—, —NR′C(S)R″—NR′C(S)NR″R′″, —SONR′R″—, —SO₂NR′R″—, —OR′, —OR′C(O)NR″R′″, —OR′C(O)OR″—, —OC(O)R′, —OC(O)NR′R″, —R′NR″C(O)R′″, —R′OR″, —R′C(O)OR″, —R′C(O)NR″R′″, —R′C(O)R″, —R′OC(O)R″, —SR′, —SOR′, —SO₂R′, —ONO₂, wherein R′, R″ and R′″ in each of the above groups may be hydrogen, hydroxyl, halogen, carboxyl, cyano, nitro, oxo (═O), thio (═S), imino (═NR′), substituted or unsubstituted alkyl, substituted or unsubstituted alkoxyl, substituted or unsubstituted alkenyl, substituted or unsubstituted alkynyl, substituted or unsubstituted cycloalkyl, substituted or unsubstituted cycloalkenyl, substituted or unsubstituted heterocyclyl, substituted or unsubstituted heterocyclyl alkyl, substituted or unsubstituted aryl, or any two of R′, R″ and R′″ may be joined to form a substituted or unsubstituted saturated or unsaturated 3- to 10-membered ring, or form oxo (═O), thio (═S) or imino (═NR′), and the ring optionally includes heteroatom(s) which may be same or different and selected from O, NRX or S. Substituents or combinations of substituents envisioned by the present invention are preferably those that result in the formation of stable or chemically feasible compounds.

The term “halo” or, alternatively, “halogen” refers to fluorine, chlorine, bromine, or iodine. The term “haloalkyl” refers to an alkyl group substituted with one or more halo groups or with combinations thereof.

### BRIEF DESCRIPTION OF THE DRAWINGS

FIG. 1 shows a synthetic route of a photoactive compound of Example 1.

FIG. 2 shows a proton nuclear magnetic resonance spectrum of Compound 1 of Example 1.

FIG. 3 shows a mass spectrum of Compound 2 of Example 1.

FIG. 4 shows a proton nuclear magnetic resonance spectrum of Compound 3 of Example 1.

FIG. 5 shows a UV-visible absorption spectrum of the photoactive compound (Compound 3) of Example 1.

FIG. 6 shows a comparison of bovine serum albumin protein immobilization efficiency of photoactive protein-immobilizing gels with concentrations of 0%, 0.75%, 1.5% and 3%.

FIG. 7 shows a comparison of bovine serum albumin protein immobilizing efficiency of photoactive protein-immobilizing gels under UV irradiation for different periods of time.

FIG. 8 shows an interaction principle between photoactive protein-immobilizing gel and protein under UV light excitation.

FIG. 9 shows a single-cell band obtained from the reaction of a photoactive protein-immobilizing gel with a protein.

### DETAILED DESCRIPTION OF THE PREFERRED EMBODIMENTS

The present invention will now be described in detail below with reference to the accompanying drawings.

General Method for Preparing Photoactive Compounds of the Present Invention

Photoactive compounds according to the present invention can be prepared by the method below. Unless otherwise indicated, variables (e.g., R₁, R₂ and R₃), when used in the following formulae, are to be understood to represent those groups described above with respect to formula (I).

A general synthetic route of the photoactive compounds is illustrated by the following.

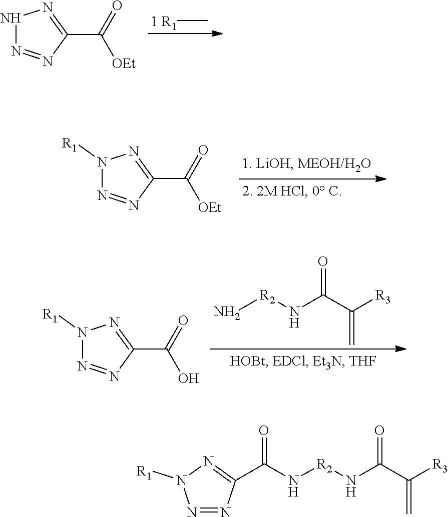

A specific synthesis scheme may involve the following:

(1) An R₁-containing monomer, (diacetoxyiodo)benzene and trifluoroethanol are weighed at a certain molar ratio. The R₁-containing monomer is first dissolved in trifluoroethanol, (diacetoxyiodo)benzene is second added thereto at −40° C., and then the mixture is stirred for a period of time under nitrogen protection. The stirred mixture is concentrated to an oil and dissolved in dichloromethane, and ethyl tetrazole-5-carboxylate, copper(II) triflate and triethylamine weighed at a certain molar ratio are added thereto, and then the mixture is stirred at room temperature under nitrogen protection for a period of time. The resulting substance is washed separately with a saturated ammonium chloride solution and brine, and then is dried with anhydrous magnesium sulfate. Finally, it is filtered and further purified by silica gel chromatography (using PE/EA as an eluent), which results in an intermediate product with a structure of formula (Ia) below.

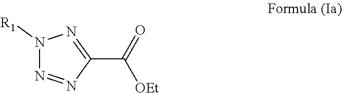

(2) The intermediate product and lithium hydroxide are weighed at a certain mass ratio. The intermediate product is then dissolved in a 1:1 (v/v) MeOH/H₂O solution, and lithium hydroxide is added thereto at 0° C. The system is stirred at room temperature under nitrogen protection to allow the reaction to proceed for a period of time. Finally, the temperature is lowered to 0° C., and the pH is adjusted to a desired value by adding HCl, which results in a mixed solution. An organic phase is then extracted from the mixed solution with an ethyl acetate solution, washed with brine, dried with anhydrous sodium sulfate, filtered and condensed to a solid, which is an electron-withdrawing group containing tetrazolyl compound with a structure of formula (Ib) below.

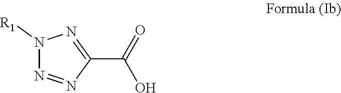

(3) An R₂ and R₃-containing compound of formula (Ic) below, 1-hydroxybenzotriazole and N-(3-aminopropyl)methacrylamide hydrochloride are weighed at a certain mass ratio. N-(3-aminopropyl)methacrylamide hydrochloride is first dissolved in tetrahydrofuran, followed by addition thereto of the compound of formula (Ic) and 1-hydroxybenzotriazole, and the reaction is run for a period of time. The electron-withdrawing group containing tetrazolyl compound (formula (Ib)) and triethylamine are weighed at a certain mass ratio of N-(3-aminopropyl)methacrylamide hydrochloride to the electron-withdrawing group containing tetrazolyl compound to triethylamine, added to the reaction mixture, and mixed therewith by stirring. The reaction is then run under reflux for a period of time. After the reaction completes, a product thereof is purified by preparative HPLC to produce, as a final product, a photoactive compound with a structure of formula (I) below.

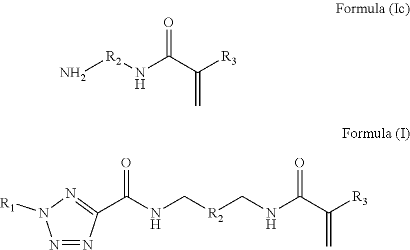

Further description is made below by reference to specific Examples.

### Example 1

After methyl-1H-pyrrole, (diacetoxyiodo)benzene and trifluoroethanol were weighed at a molar ratio of 1:11:200, methyl-1H-pyrrole was first dissolved in trifluoroethanol, and (diacetoxyiodo)benzene was added thereto at −40° C., and then the mixture was stirred under nitrogen protection for 3 h. The stirred mixture was concentrated to a black oil and dissolved in dichloromethane. After that, ethyl tetrazole-5-carboxylate, copper(II) triflate and triethylamine weighed at a molar ratio of 3:1:13 were added thereto, and then the mixture was stirred at room temperature under nitrogen protection for 24 h. The resulting substance was washed separately with a saturated ammonium chloride solution and brine, and then was dried with anhydrous magnesium sulfate. Finally, it was filtered and further purified by silica gel chromatography (using PE/EA=8:1 as an eluent), which resulted in a brown oil-like intermediate product (Compound 1) with a structure of the formula below.

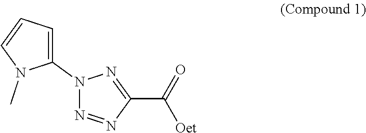

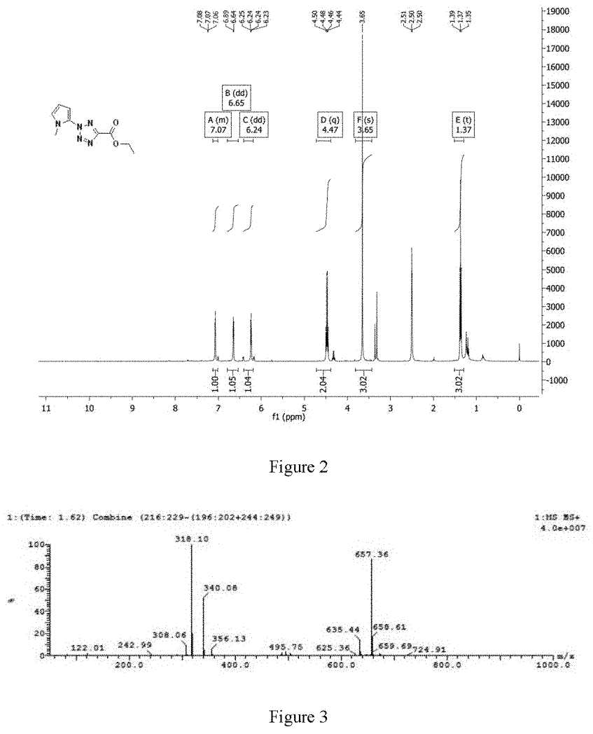

The yield of the above preparation approach was 9.6%. As shown in FIG. 2, proton nuclear magnetic resonance data of Compound 1 were as follows: 1H NMR (400 MHz, DMSO): δ 7.13-7.00 (m, 1H), 6.65 (dd, J=3.9, 1.9 Hz, 1H), 6.24 (dd, J=3.9, 3.0 Hz, 1H), 4.47 (q, J=7.1 Hz, 2H), 3.65 (s, 3H), 1.37 (t, J=7.1 Hz, 3H). Its mass spectrometric analysis suggested [M+H]+ 222.0.

The intermediate product and lithium hydroxide were weighed at a mass ratio of 1:1.8. The intermediate product was then dissolved in a 20 ml 1:1 (v/v) MeOH/H₂O solution, and lithium hydroxide was added thereto at 0° C. The system was set to room temperature and stirred under nitrogen protection to allow the reaction to proceed for 1 h. Finally, the temperature was lowered to 0° C., and the pH was adjusted to 7 by adding 2N HCl, which resulted in a mixed solution. An organic phase was then extracted from the mixed solution with an ethyl acetate solution, washed with brine, dried with anhydrous sodium sulfate, filtered and condensed to a brown solid, which was an electron-withdrawing group containing tetrazolyl compound with a structure of the following formula (Compound 2). A final product of 370 mg was obtained in a yield of 94%. Mass spectrometric analysis of Compound 2 suggested [M+H]+ 194.1.

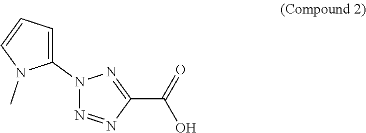

1-(3-Dimethylaminopropyl)-3-ethylcarbodiimide hydrochloride, 1-hydroxybenzotriazole and N-(3-aminopropyl)methacrylamide hydrochloride were weighed at a mass ratio of 2:1:1. N-(3-aminopropyl)methacrylamide hydrochloride was first dissolved in 50 mL tetrahydrofuran, followed by addition thereto of 1-(3-dimethylaminopropyl)-3-ethylcarbodiimide hydrochloride and 1-hydroxybenzotriazole at 0° C., and the reaction was run for 1 h. The electron-withdrawing group containing tetrazolyl compound (Compound 2) and triethylamine were weighed at a mass ratio of N-(3-aminopropyl)methacrylamide hydrochloride to the electron-withdrawing group containing tetrazolyl compound (Compound 2) to triethylamine of 1:1:2.5, added to the reaction mixture, and mixed therewith by stirring. The reaction was then run for 12 h under reflux. After the reaction completed, a product thereof was purified by preparative HPLC to produce, as a final product, a photoactive compound (Compound 3) with a structure of the following formula in the form of a white power which weighed 41 mg (in a yield of 6.7%).

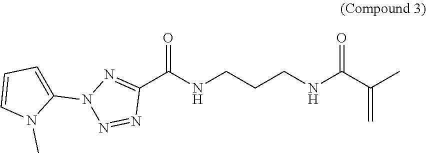 [SMILES: C=C(C)C(=O)NCCCNC(=O)c1nnn(-c2cccn2C)n1]

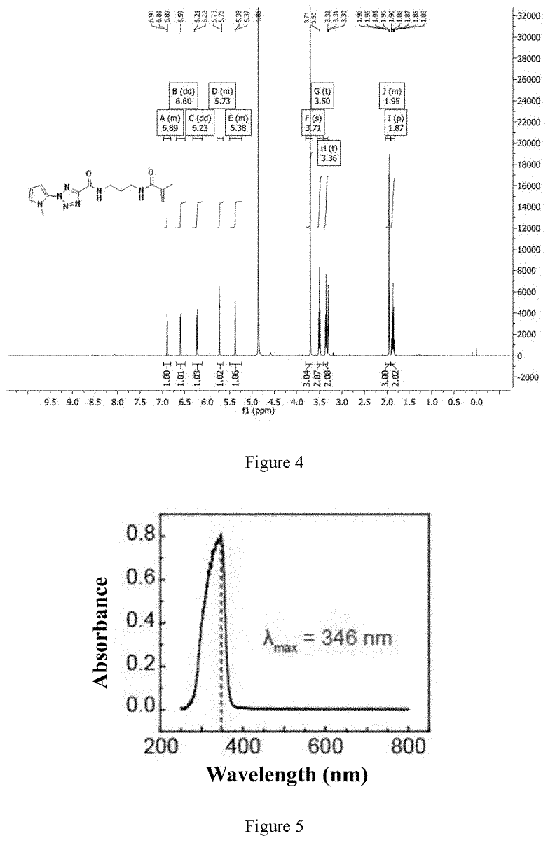

As shown in FIGS. 3 to 4, proton nuclear magnetic resonance data of Compound 3 were as follows: 1H NMR (400 MHz, MeOD) δ 6.97-6.82 (m, 1H), 6.60 (dd, J=4.0, 1.9 Hz, 1H), 6.23 (dd, J=3.9, 3.0 Hz, 1H), 5.78-5.64 (m, 1H), 5.50-5.23 (m, 1H), 3.71 (s, 3H), 3.50 (t, J=6.8 Hz, 2H), 3.36 (t, J=6.7 Hz, 2H), 2.03-1.92 (m, 3H), 1.87 (p, J=6.7 Hz, 2H). Mass spectrometric analysis of this photoactive compound suggested [M+H]+ 318.1.

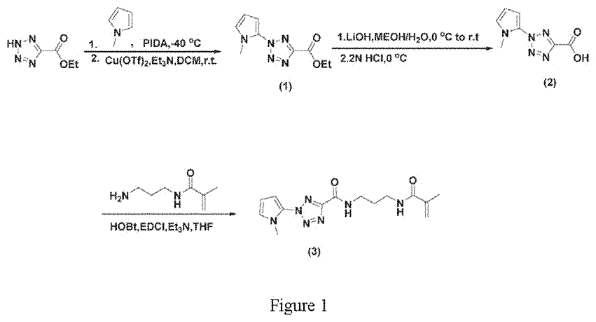

The synthetic route of the photoactive compound of this Example is shown in FIG. 1. FIG. 5 shows a UV-Visible absorption spectrum of the photoactive compound of this Example, which indicates the light absorption characterization of the compound by an Evolution 220 UV-Vis spectrophotometer with a spectral scan range of 300 nm to 800 nm from Agilent Technologies (China) Co., Ltd. Longer excitation wavelength in a narrower UV spectral band means lower energy, which avoids UV-induced deactivation of a protein's antigenic site and reduces UV-induced background autofluorescence from the gel itself.

### Example 2

A photoactive compound with a structure of the following formula (Compound 4) was prepared.

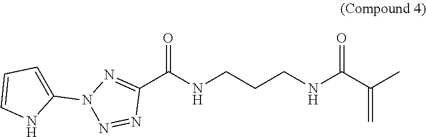 [SMILES: C=C(C)C(=O)NCCCNC(=O)c1nnn(-c2ccc[nH]2)n1]

Pyrrole, (diacetoxyiodo)benzene and trifluoroethanol were weighed at a molar ratio of 1:10:170. Pyrrole was first dissolved in trifluoroethanol, and (diacetoxyiodo)benzene was added thereto at −40° C., and then the mixture was stirred under nitrogen protection for 3 h. The stirred mixture was concentrated to a black oil and dissolved in dichloromethane. Subsequently, ethyl tetrazole-5-carboxylate, copper(II) triflate and triethylamine weighed at a molar ratio of 4:1:15 were added thereto, and then the mixture was stirred at room temperature under nitrogen protection for 24 h. The resulting substance was washed separately with a saturated ammonium chloride solution and brine, and then was dried with anhydrous magnesium sulfate. Finally, it was filtered and further purified by silica gel chromatography (using PE:EA=7:1 as an eluent), which resulted in a brown oil-like intermediate product.

After the intermediate product and lithium hydroxide were weighed at a mass ratio of 1:1, the intermediate product was first dissolved in a 20 ml 1:1 (v/v) MeOH/H₂O solution, and lithium hydroxide was added thereto at 0° C. The system was set to room temperature and stirred under nitrogen protection to allow the reaction to proceed for 2 h. Finally, the temperature was lowered to 0° C., and the pH was adjusted to 7.5 by adding 2N HCl, which resulted in a mixed solution. An organic phase was then extracted from the mixed solution with an ethyl acetate solution, washed with brine, dried with anhydrous sodium sulfate, filtered and condensed to a brown solid, which was an electron-withdrawing group containing tetrazolyl compound.

1-(3-Dimethylaminopropyl)-3-ethylcarbodiimide hydrochloride, 1-hydroxybenzotriazole and N-(3-aminopropyl)methacrylamide hydrochloride were weighed at a mass ratio of 3:1:1. N-(3-aminopropyl)methacrylamide hydrochloride was first dissolved in 50 mL tetrahydrofuran, followed by addition thereto of 1-(3-dimethylaminopropyl)-3-ethylcarbodiimide hydrochloride and 1-hydroxybenzotriazole at 0° C., and the reaction was run for 30 min. The electron-withdrawing group containing tetrazolyl compound and triethylamine were weighed at a mass ratio of N-(3-aminopropyl)methacrylamide hydrochloride to the electron-withdrawing group containing tetrazolyl compound to triethylamine of 1:1:2, added to the reaction mixture, and mixed therewith by stirring. The reaction was then run for 10 h under reflux. After the reaction completed, a product thereof was purified by preparative HPLC to produce, as a final product, a photoactive compound (Compound 4) in the form of a white power.

### Example 3

A photoactive compound with a structure of the following formula (Compound 5) was prepared.

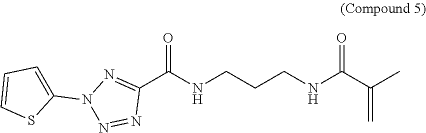 [SMILES: C=C(C)C(=O)NCCCNC(=O)c1nnn(-c2cccs2)n1]

Thiophene, (diacetoxyiodo)benzene and trifluoroethanol were weighed at a molar ratio of 1:12:150. Thiophene was first dissolved in trifluoroethanol, and (diacetoxyiodo)benzene was added thereto at −40° C., and then the mixture was stirred under nitrogen protection for 4 h. The stirred mixture was concentrated to a black oil and dissolved in dichloromethane. Subsequently, ethyl tetrazole-5-carboxylate, copper(II) triflate and triethylamine weighed at a molar ratio of 5:1:16 were added thereto, and then the mixture was stirred at room temperature under nitrogen protection for 27 h. The resulting substance was washed with a saturated ammonium chloride solution, and then was dried with anhydrous magnesium sulfate. Finally, it was filtered and further purified by silica gel chromatography (using PE:EA=6:1 as an eluent), which resulted in a brown oil-like intermediate product.

After the intermediate product and lithium hydroxide were weighed at a mass ratio of 1:2, the intermediate product was first dissolved in a 20 ml 1:1 (v/v) MeOH/H₂O solution, and lithium hydroxide was added thereto at 0° C. The system was set to room temperature and stirred under nitrogen protection to allow the reaction to proceed for 1.5 h. Finally, the temperature was lowered to 0° C., and the pH was adjusted to 8 by adding 2N HCl, which resulted in a mixed solution. An organic phase was then extracted from the mixed solution with an ethyl acetate solution, washed with brine, dried with anhydrous sodium sulfate, filtered and condensed to a brown solid, which was an electron-withdrawing group containing tetrazolyl compound. 1-(3-Dimethylaminopropyl)-3-ethylcarbodiimide hydrochloride, 1-hydroxybenzotriazole and N-(3-aminopropyl)methacrylamide hydrochloride were weighed at a mass ratio of 5:2:1. N-(3-aminopropyl)methacrylamide hydrochloride was first dissolved in 50 mL tetrahydrofuran, followed by addition thereto of 1-(3-dimethylaminopropyl)-3-ethylcarbodiimide hydrochloride and 1-hydroxybenzotriazole at 0° C., and the reaction was run for 2 h. The electron-withdrawing group containing tetrazolyl compound and triethylamine were weighed at a mass ratio of N-(3-aminopropyl)methacrylamide hydrochloride to the electron-withdrawing group containing tetrazolyl compound to triethylamine of 1:1:4, added to the reaction mixture, and mixed therewith by stirring. The reaction was then run for 15 h under reflux. After the reaction completed, a product thereof was purified by preparative HPLC to produce, as a final product, a photoactive compound (Compound 5) in the form of a white power.

### Example 4

A photoactive compound with a structure of the following formula (Compound 6) was prepared.

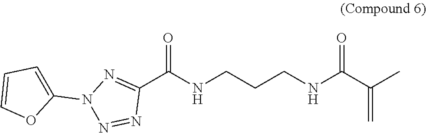 [SMILES: C=C(C)C(=O)NCCCNC(=O)c1nnn(-c2ccco2)n1]

Furan, (diacetoxyiodo)benzene and trifluoroethanol were weighed at a molar ratio of 1:13:130. Furan was first dissolved in trifluoroethanol, and (diacetoxyiodo)benzene was added thereto at −40° C., and then the mixture was stirred under nitrogen protection for 4 h. The stirred mixture was concentrated to a black oil and dissolved in dichloromethane. Afterwards, ethyl tetrazole-5-carboxylate, copper(II) triflate and triethylamine weighed at a molar ratio of 7:1:18 were added thereto, and then the mixture was stirred at room temperature under nitrogen protection for 30 h. The resulting substance was washed separately with a saturated ammonium chloride solution and brine, and then was dried with anhydrous sodium sulfate. Finally, it was filtered and further purified by silica gel chromatography (using PE:EA=5:1 as an eluent), which resulted in a brown oil-like intermediate product.

After the intermediate product and lithium hydroxide were weighed at a mass ratio of 1:3, the intermediate product was first dissolved in a 20 ml 1:1 (v/v) MeOH/H₂O solution, and lithium hydroxide was added thereto at 0° C. The system was set to room temperature and stirred under nitrogen protection to allow the reaction to proceed for 2.5 h. Finally, the temperature was lowered to 0° C., and the pH was adjusted to 8.5 by adding 2N HCl, which resulted in a mixed solution. An organic phase was then extracted from the mixed solution with an ethyl acetate solution, washed separately with a saturated ammonium chloride solution and brine, dried with anhydrous sodium sulfate, filtered and condensed to a brown solid, which was an electron-withdrawing group containing tetrazolyl compound.

1-(3-Dimethylaminopropyl)-3-ethylcarbodiimide hydrochloride, 1-hydroxybenzotriazole and N-(3-aminopropyl)methacrylamide hydrochloride were weighed at a mass ratio of 6:2:1. N-(3-aminopropyl)methacrylamide hydrochloride was first dissolved in 50 mL tetrahydrofuran, followed by addition thereto of 1-(3-dimethylaminopropyl)-3-ethylcarbodiimide hydrochloride and 1-hydroxybenzotriazole at 0° C., and the reaction was run for 1.5 h. The electron-withdrawing group containing tetrazolyl compound and triethylamine were weighed at a mass ratio of N-(3-aminopropyl)methacrylamide hydrochloride to the electron-withdrawing group containing tetrazolyl compound to triethylamine of 1:1:6, added to the reaction mixture, and mixed therewith by stirring. The reaction was then run for 17 h under reflux. After the reaction completed, a product thereof was purified by preparative HPLC to produce, as a final product, a photoactive compound (Compound 6) in the form of a white power.

### Example 5

A photoactive compound with a structure of the following formula (Compound 7) was prepared.

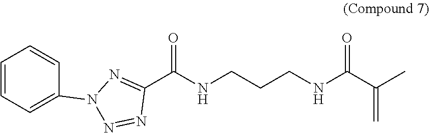 [SMILES: C=C(C)C(=O)NCCCNC(=O)c1nnn(-c2ccccc2)n1]

Toluene, (diacetoxyiodo)benzene and trifluoroethanol were weighed at a molar ratio of 1:15:100. Toluene was first dissolved in trifluoroethanol, and (diacetoxyiodo)benzene was added thereto at −40° C., and then the mixture was stirred under nitrogen protection for 5 h. The stirred mixture was concentrated to a black oil and dissolved in dichloromethane. Thereafter, ethyl tetrazole-5-carboxylate, copper(II) triflate and triethylamine weighed at a molar ratio of 8:1:20 were added thereto, and then the mixture was stirred at room temperature under nitrogen protection for 30 h. The resulting substance was washed separately with a saturated ammonium chloride solution and brine, and then was dried with anhydrous magnesium sulfate. Finally, it was filtered and further purified by silica gel chromatography (using PE:EA=4:1 as an eluent), which resulted in a brown oil-like intermediate product.

After the intermediate product and lithium hydroxide were weighed at a mass ratio of 1:5, the intermediate product was first dissolved in a 20 ml 1:1 (v/v) MeOH/H₂O solution, and lithium hydroxide was added thereto at 0° C. The system was set to room temperature and stirred under nitrogen protection to allow the reaction to proceed for 2 h. Finally, the temperature was lowered to 0° C., and the pH was adjusted to 9 by adding 2N HCl, which resulted in a mixed solution. An organic phase was then extracted from the mixed solution with an ethyl acetate solution, washed with brine, dried with anhydrous magnesium sulfate, filtered and condensed to a brown solid, which was an electron-withdrawing group containing tetrazolyl compound.

1-(3-Dimethylaminopropyl)-3-ethylcarbodiimide hydrochloride, 1-hydroxybenzotriazole and N-(3-aminopropyl)methacrylamide hydrochloride were weighed at a mass ratio of 7:3:1. N-(3-aminopropyl)methacrylamide hydrochloride was first dissolved in 50 mL tetrahydrofuran, followed by addition thereto of 1-(3-dimethylaminopropyl)-3-ethylcarbodiimide hydrochloride and 1-hydroxybenzotriazole at 0° C., and the reaction was run for 1 h. The electron-withdrawing group containing tetrazolyl compound and triethylamine were weighed at a mass ratio of N-(3-aminopropyl)methacrylamide hydrochloride to the electron-withdrawing group containing tetrazolyl compound to triethylamine of 1:1:8, added to the reaction mixture, and mixed therewith by stirring. The reaction was then run for 20 h under reflux. After the reaction completed, a product thereof was purified by preparative HPLC to produce, as a final product, a photoactive compound (Compound 7) in the form of a white power.

### Example 6

A photoactive compound with a structure of the following formula (Compound 8) was prepared.

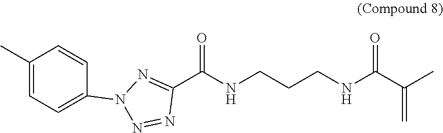 [SMILES: C=C(C)C(=O)NCCCNC(=O)c1nnn(-c2ccc(C)cc2)n1]

p-Xylene, (diacetoxyiodo)benzene and trifluoroethanol were weighed at a molar ratio of 1:15:100. p-Xylene was first dissolved in trifluoroethanol, and (diacetoxyiodo)benzene was added thereto at −40° C., and then the mixture was stirred under nitrogen protection for 5 h. The stirred mixture was concentrated to a black oil and dissolved in dichloromethane. Thereafter, ethyl tetrazole-5-carboxylate, copper(II) triflate and triethylamine weighed at a molar ratio of 8:1:20 were added thereto, and then the mixture was stirred at room temperature under nitrogen protection for 30 h. The resulting substance was washed separately with a saturated ammonium chloride solution and brine, and then was dried with anhydrous magnesium sulfate. Finally, it was filtered and further purified by silica gel chromatography (using PE:EA=4:1 as an eluent), which resulted in a brown oil-like intermediate product.

After the intermediate product and lithium hydroxide were weighed at a mass ratio of 1:5, the intermediate product was first dissolved in a 20 ml 1:1 (v/v) MeOH/H₂O solution, and lithium hydroxide was added thereto at 0° C. The system was set to room temperature and stirred under nitrogen protection to allow the reaction to proceed for 2 h. Finally, the temperature was lowered to 0° C., and the pH was adjusted to 9 by adding 2N HCl, which resulted in a mixed solution. An organic phase was extracted from the mixed solution with an ethyl acetate solution, washed with brine, dried with anhydrous magnesium sulfate, filtered and condensed to a brown solid, which was an electron-withdrawing group containing tetrazolyl compound. 1-(3-Dimethylaminopropyl)-3-ethylcarbodiimide hydrochloride, 1-hydroxybenzotriazole and N-(3-aminopropyl)methacrylamide hydrochloride were weighed at a mass ratio of 7:3:1. N-(3-aminopropyl)methacrylamide hydrochloride was first dissolved in 50 mL tetrahydrofuran, followed by addition thereto of 1-(3-dimethylaminopropyl)-3-ethylcarbodiimide hydrochloride and 1-hydroxybenzotriazole at 0° C., and the reaction was run for 1 h. The electron-withdrawing group containing tetrazolyl compound and triethylamine were weighed at a mass ratio of N-(3-aminopropyl)methacrylamide hydrochloride to the electron-withdrawing group containing tetrazolyl compound to triethylamine of 1:1:2, added to the reaction mixture, and mixed therewith by stirring. The reaction was then run for 12 h under reflux. After the reaction completed, a product thereof was purified by preparative HPLC to produce, as a final product, a photoactive compound (Compound 8) in the form of a white power.

### Example 7

A photoactive protein-immobilizing gel containing the photoactive compound of Example 1 as a photoactive ingredient was prepared.

The photoactive compound of Example 1 (Compound 3) was dissolved in dimethyl sulfoxide to produce a 100 mM storage solution. In each of four 1.5 mL Ep tubes, 25 μL of a 1.5 M Tris-HCl buffer (pH=8.8), 166.7 μL of a 30% acrylamide/bis-acrylamide (29:1) solution and 265.3 μL of ddH₂O were added, and 15 μL, 7.5 μL, 3.75 μL and 0 μL of the storage solution were then respectively further added therein, which resulted in gel precursor solutions at respective concentrations of 3%, 1.5%, 0.75% and 0%. After that, each Ep tube was further added therein with 10 μL 5% SDS, 10 μL 5% Triton X-100, 4 μL ammonium persulphate (APS) and 4 μL tetramethylethylenediamine (TEMED) and gently shaken until homogenous mixing was achieved. The resulting solutions were then dropped onto porous microarray molds and slowly and gently covered with glass slides to avoid the occurrence of bubbles. After the setups were left at rest for 20 minutes, the polyacrylamide hydrogels cured and the molds were peeled off. In this way, at least four sets of porous microarray gels with different concentrations were obtained.

1 L of an electrophoresis buffer was prepared by weighing and mixing 500 mg 0.5% SDS, 100 μL 0.1% v/v Triton X-100, 250 mg 0.25% sodium deoxycholate, 1.514 g Tris and 7.2 g glycine and adjusting the pH to 8.3, and then stored at 4° C. This electrophoresis buffer was heated in a water bath to 55° C., and a UV light source was activated in advance to stabilize the light source.

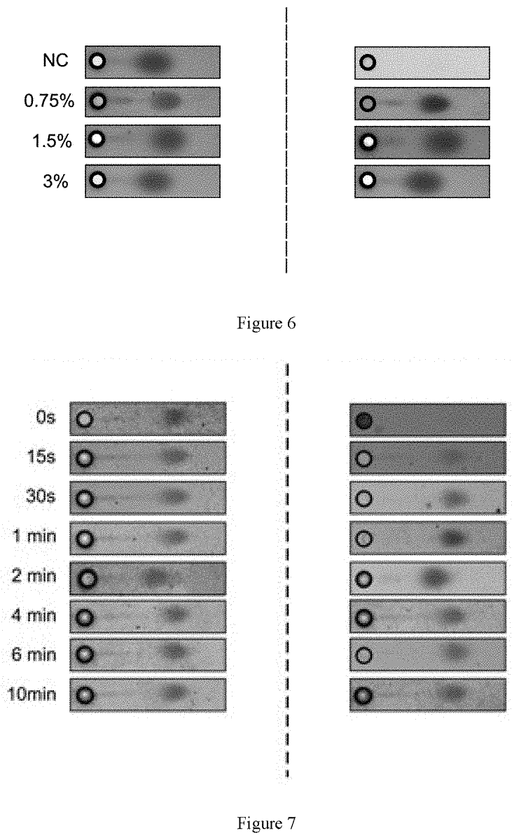

Each glass slide with a gel coated thereon was placed in a clean vessel, with the gel-coated surface facing upward. A 5.12 mg/mL bovine serum albumin protein (BSA) solution in an amount of 200 μL was dropped thereon, and the glass slide was gently shaken to allow the BSA solution to evenly spread over the gel surface and then left at rest for 3 min. The gel was placed in an electrophoresis tank, and 10 mL of the electrophoresis buffer that had been preheated was gently and quickly poured into the electrophoresis tank at a corner thereof. Immediately after that, the power supply was turned on to apply a voltage of 200 V (E=40 v/cm²). After the electrophoresis was run 30 s for protein separation, the power supply was immediately turned off, and the gel was exposed to UV light for 10 min. Following the exposure, the gel was taken out and stained in a Coomassie brilliant blue solution (a mixed solution of 0.1 g of a Coomassie brilliant blue powder in 20 mL methanol, 16 mL water and 4 mL acetic acid) for 5 min. At this point, photographs were taken to record protein immobilization conditions of the four sets of gels (the left column in FIG. 6). Each gel was then washed under being shaken in a TBST buffer (100 mM Tris titrated to pH 7.5 with HCl, 150 mM NaCl, 0.1% Tween 20, 9480, EMD Millipore). The buffer was replaced with a fresh volume every 15 min in the first 2 hours, and the gel was again washed under being shaken in a TBST buffer for 12 h. Thereafter, photographs were again taken to record protein immobilization conditions of the four sets of gels (the right column in FIG. 6), as shown in FIG. 6. As can be seen from this figure, in contrast to the case without using a photoactive protein-immobilizing gel, where the protein was almost totally washed away within 12 h, even the low-concentration photoactive protein-immobilizing gel, i.e., the 0.75% photoactive protein-immobilizing gel, was demonstrated to immobilize the protein therein with high efficiency when excited by UV light.

In this Example, photoactive protein-immobilizing gels of the same concentration, each containing bovine serum albumin proteins of same concentration, were excited by UV light for different periods of time of 0 s, 15 s, 30 s, 1 min, 2 min, 4 min, 6 min and 10 min and each underwent the same shaken wash procedure as defined above, and the results are shown in FIG. 7. In this figure, the protein immobilization conditions after electrophoresis are shown in the left column, and the protein immobilization conditions after UV irradiation of different periods of time are shown in the right column. Even 15 s of UV excitation could trigger a protein-immobilizing effect, and an even stronger immobilizing effect was obtained when the period of excitation was extended to 30 s or longer. This demonstrates that the photoactive protein-immobilizing gel of the present invention requires a shorter period of UV excitation, which avoids deactivation of a protein's antigenic site and reduces UV-induced background autofluorescence from the gel itself.

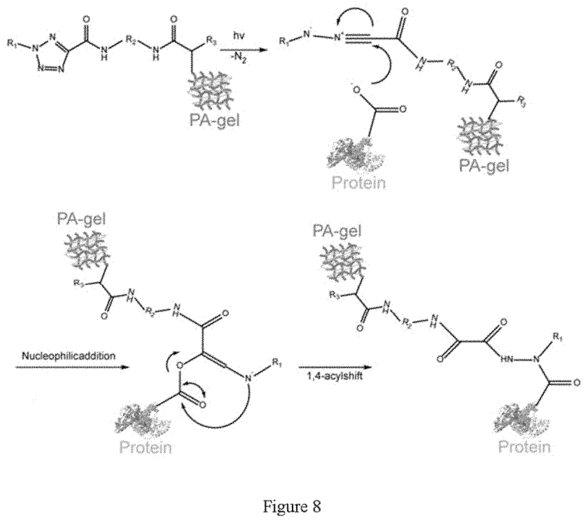

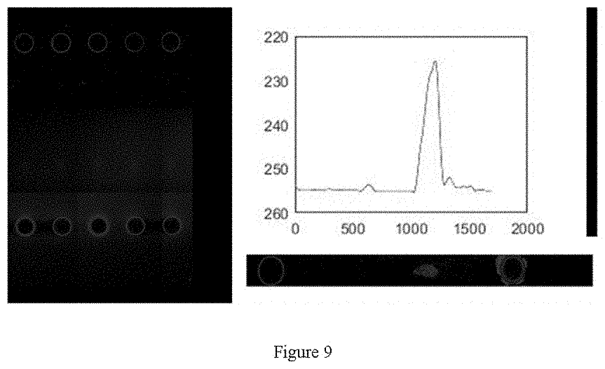

FIG. 8 shows an interaction principle between a photoactive protein-immobilizing gel and a protein under UV light excitation. As can be seen from the figure, when the photoactive protein-immobilizing gel is excited by UV light with a wavelength of 300-400 nm, the tetrazolyl ring in the photoactive compound is split up and undergoes a nucleophilic addition reaction with a carboxyl group of the protein. Under the action of the electron-withdrawing group, 1,4-acyl group migrates, which eventually results in a stable structure between the gel and the protein, and thus immobilizes the protein. Therefore, the photoactive protein-immobilizing gel of the present invention can be excited within a short period of time, which is commonly within 30 s, when exposed to UV light. That is, it can more quickly polymerize with functional groups in protein molecules. This can avoid protein diffusion which may lead to a reduction in sensitivity due to a slow polymerization rate, which significantly promotes protein immobilizing efficiency. Moreover, since the photoactive protein-immobilizing gel reacts with carboxyl groups in proteins, it is not selective for any particular protein and can be used to immobilize all proteins. The photoactive protein-immobilizing gel of the present invention not only does not affect the molecular sieving of SDS-PAGE used in electrophoresis, which allows the method to retain its own specificity and separation resolution, and also the non-selectively immobilization of all proteins. Therefore, it can better work with electrophoretic separation and facilitate subsequent protein detection. FIG. 9 shows single-cell bands. Single cells placed in wells lysed and underwent electrophoresis. After the electrophoresis was completed, the photoactive protein-immobilizing gel reacted with proteins under UV excitation, and in-situ immobilized the proteins within the gel. Incubation was carried out with a primary antibody that could recognize a particular protein, followed by the use of a fluorescent secondary antibody to recognize the primary antibody. Finally, fluorescent band was detected. The protein under assay in the figure was the internal reference protein Tubulin. Obvious fluorescent bands were visible in the left half of the figure, which indicated no impact of the immobilized proteins on the antigen-antibody binding. A notable signal peak was detected in data analysis of Matlab, and an area under the curve (peak) represented an amount of the detected antigen. As can be seen from the figure, protein immobilization by the photoactive protein-immobilizing gel of the present invention does not affect antigen-antibody binding, receptor-ligand binding, enzymatic activity, aptamer binding, or the like in subsequent detection, and thus does not affect subsequent establishment of a detection system.

In the present invention, washing agents include saturated ammonium chloride and brine; drying agents include anhydrous magnesium sulfate and anhydrous sodium sulfate; purification processes involve the use of silica gel chromatography (using PE/EA=4-8:1 as an eluent) and preparative HPLC; and organic solvents include one or more of trifluoroethanol, dichloromethane, tetrahydrofuran or N,N-dimethylformamide. The foregoing technical features of the various embodiments may be combined in any way. Although not all such combinations have been described above for the sake of brevity, any of them is considered to fall within the scope of this specification as long as there is no contradiction between the technical features.

The embodiments disclosed herein are merely intended to illustrate the present invention without limiting the scope of the appended claims. Other substantively equivalent alternatives conceivable by those skilled in the art are all within the scope of the invention sought to be protected hereby.

1. A photoactive compound, wherein the photoactive compound has a structure of formula (I): 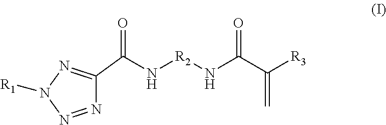
wherein: R₁ is selected from nitro, cyano, halogen, haloalkyl, —ORₐ, —S(═O)_{q}—Rₐ, —C(═Y)—Rₐ, —NRₐR_{b}, substituted or unsubstituted heterocyclyl and substituted or unsubstituted aryl, wherein Rₐ and R_{b} of each occurrence in the R₁ are same or different and independently selected from hydrogen, nitro, hydroxyl, cyano, halogen, substituted or unsubstituted alkyl and substituted or unsubstituted cycloalkyl;
R₂ is selected from —NH—, —O—, —S—, —C(═Y)—, —C(═Y)—CRₐR_{b}—, —CRₐR_{b}—C(═Y)—CRₐR_{b}—, —Y—CRₐR_{b}—, —CRₐR_{b}—Y—CRₐR_{b}—, —C(═Y)—NRₐ—, —NRₐ—C(═Y)—NRₐ—, —S(═O)_{q}—NRₐ—, —NRₐ—S(═O)_{q}—NRₐ—, —S(═O)_{q}—CRₐR_{b}—, —CRₐR_{b}—S(═O)_{q}—CRₐR_{b}—, substituted or unsubstituted alkyl, substituted or unsubstituted alkenyl, substituted or unsubstituted alkynyl, substituted or unsubstituted cycloalkyl, substituted or unsubstituted heterocyclyl and substituted or unsubstituted aryl, wherein Rₐ and R_{b} of each occurrence in R₂ are same or different and independently selected from hydrogen, nitro, hydroxyl, cyano, halogen, substituted or unsubstituted C₁₋₆ alkyl, substituted or unsubstituted C₂₋₆ alkenyl, substituted or unsubstituted C₂₋₆ alkynyl, substituted or unsubstituted C₃₋₆ cycloalkyl and substituted or unsubstituted C₃₋₆ cycloalkenyl;
R₃ is selected from hydrogen, —NRₐR_{b}, —C(═Y)—Rₐ, —CRₐR_{b}—C(═Y)—Rₐ, —CRₐR_{b}—Y—Rₐ, —C(═Y)—NRₐR_{b}, —NRₐ—C(═Y)—NRₐR_{b}, —S(═O)_{q}—NRₐR_{b}, —NRₐ—S(═O)_{q}—NRₐR_{b}, —S(═O)_{q}—Rₐ, —CRₐR_{b}—S(═O)_{q}—Rₐ, substituted or unsubstituted alkyl, substituted or unsubstituted cycloalkyl, substituted or unsubstituted heterocyclyl and substituted or unsubstituted aryl, wherein Rₐ and R_{b} of each occurrence in R₃ are same or different and independently selected from hydrogen, nitro, hydroxyl, cyano, halogen, substituted or unsubstituted alkyl and substituted or unsubstituted cycloalkyl;
Y of each occurrence is independently selected from O, S and NRₐ; and
q of each occurrence independently represents 0, 1 or 2.

2. The photoactive compound of claim 1, wherein R₁ is selected from any of following groups:
—NO₂, CN, —Cl, —F, —Br, —CH₂Cl, —CHCl₂, —CCl₃, —CH₂F, —CHF₂, —CF₃, —OCH₃, —OCH₂CH₃, —COOH, —C(═O)—CH₃, —C(═O)—CH₂CH₃, —C(═O)—CH₂CN, —C(═O)—CH₂NO₂, —SCH₃, —S(═O)₂—OH, —S(═O)₂—CH₃, —S(═O)₂—CH₂CN, —S(═O)₂—CH₂CH₂NO₂, —NCH₃CH₃, —NHCH₃, —NHCH₂CN, —

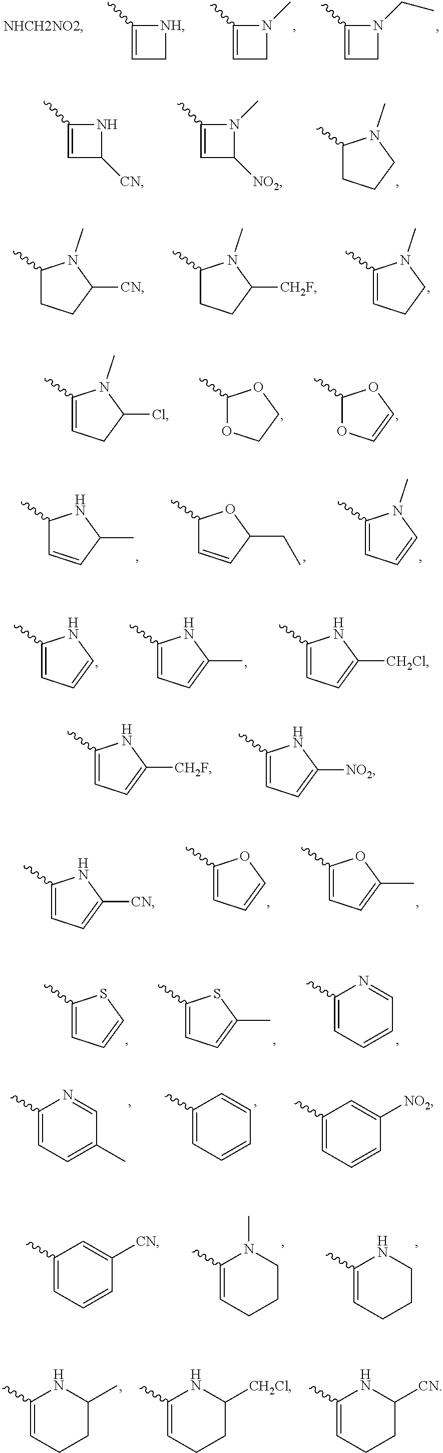

3. The photoactive compound of claim 1, wherein R₂ is selected from any of following groups:
—C(═O)—, —C(═O)—CH₂—, —CH₂—C(═O)—CH₂—, —CH₂—, —CH₂—CH₂—, —CH(CH₃)—, —CH₂—CH₂—CH₂—, —CH₂—CH(CH₃)—CH₂—, —CH═CH—, —CH═C(CH₃)—, —C(CH₃)═C(CH₃)—, —CH₂—CH═CH—CH₂—, —CH₂—NH—CH₂—, —NH—CH₂—, —S(═O)₂—CH₂—, —CH₂—S(═O)₂—CH₂—, —CH(OH)—, —CH₂CH(OH)CH₂—, —CH(CN)—, —CH₂CH(CN)CH₂—, —CH(NO₂)—, —CH₂CH(NO₂)CH₂—, —CH₂—O—CH₂—, —CH₂—S—CH₂—.

4. The photoactive compound of claim 1, wherein R₃ is selected from any of following groups:
—NH₂, —NHCH₃, —CH₃, —CH₂CH₃, —CH₂CH₂CH₃, —CH(OH)CH₃, —C(═O)—CH₃, —CH₂—C(═O)—CH₃, —C(═O)—NH₂, —CH₂—O—CH₃, —CH₂—S—CH₃, —NH—S(═O)₂—NH—CH₃, —S(═O)₂—CH₂CH₃, —CH₂—S(═O)₂—CH₃,

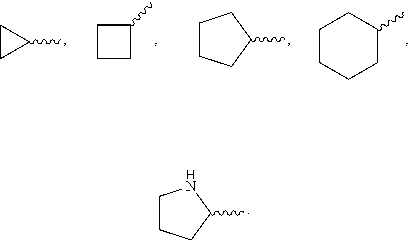

5. The photoactive compound of claim 1, wherein R₁ is substituted or unsubstituted heterocyclyl or substituted or unsubstituted aryl.

6. The photoactive compound of claim 1, wherein R₂ is substituted or unsubstituted alkyl.

7. The photoactive compound of claim 5, wherein R₂ is substituted or unsubstituted alkyl.

8. The photoactive compound of claim 1, wherein R₃ is substituted or unsubstituted alkyl.

9. The photoactive compound of claim 5, wherein R₃ is substituted or unsubstituted alkyl.

10. The photoactive compound of claim 6, wherein R₃ is substituted or unsubstituted alkyl.

11. The photoactive compound of claim 7, wherein R₃ is substituted or unsubstituted alkyl.

12. The photoactive compound of claim 1, wherein R₂ is substituted or unsubstituted C₁₋₆ alkyl and R₃ is substituted or unsubstituted C₁₋₆ alkyl.

13. The photoactive compound of claim 5, wherein R₂ is substituted or unsubstituted C₁₋₆ alkyl and R₃ is substituted or unsubstituted C₁₋₆ alkyl.

14. The photoactive compound of claim 12, wherein R₁ is selected from any of following groups:

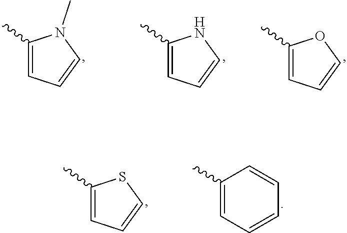

15. The photoactive compound of claim 14, wherein R₂ is unsubstituted C₁₋₆ alkyl.

16. The photoactive compound of claim 14, wherein R₃ is unsubstituted C₁₋₆ alkyl.

17. The photoactive compound of claim 1, which is selected from any of following compounds:

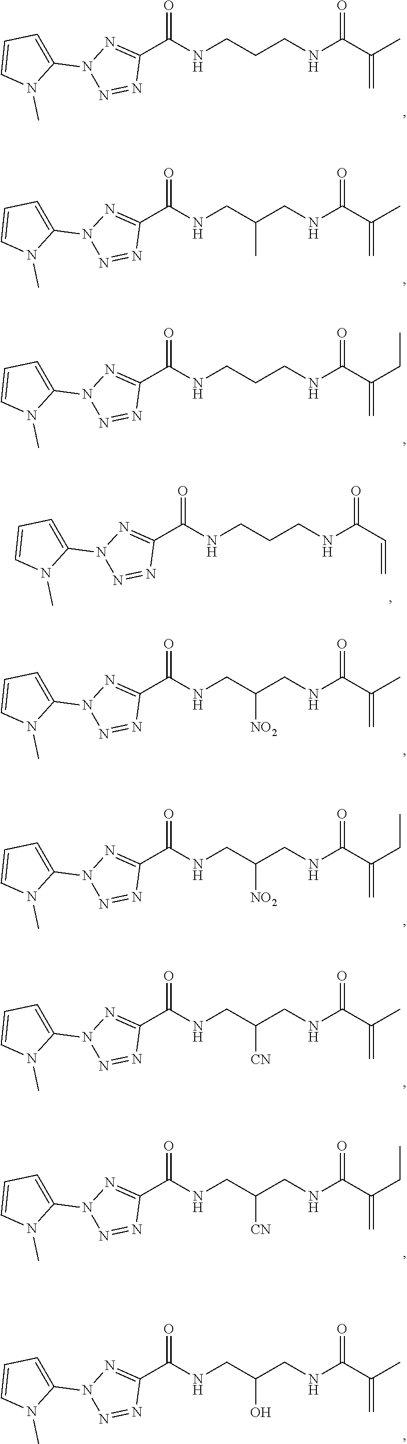

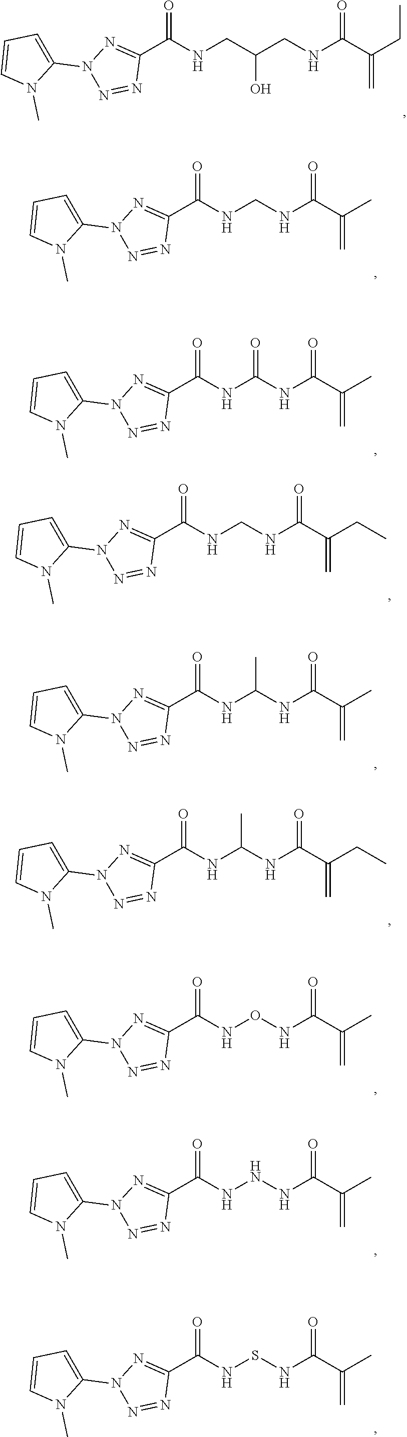

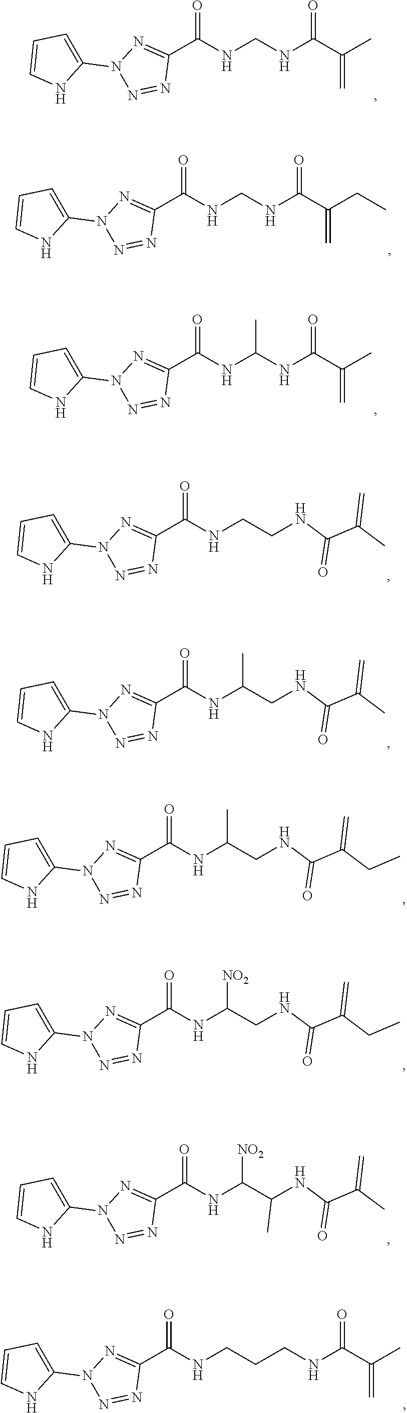

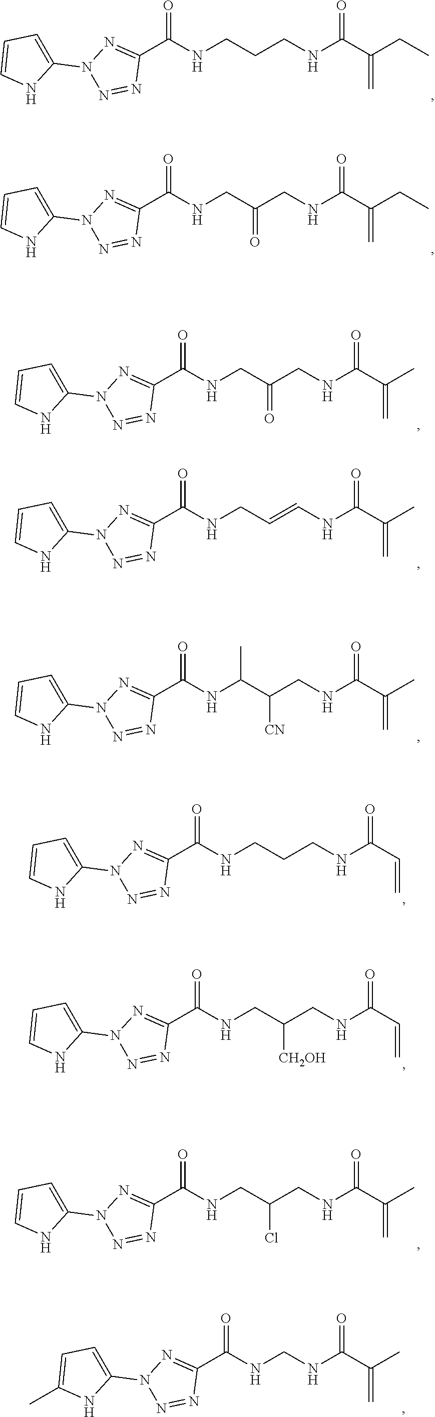

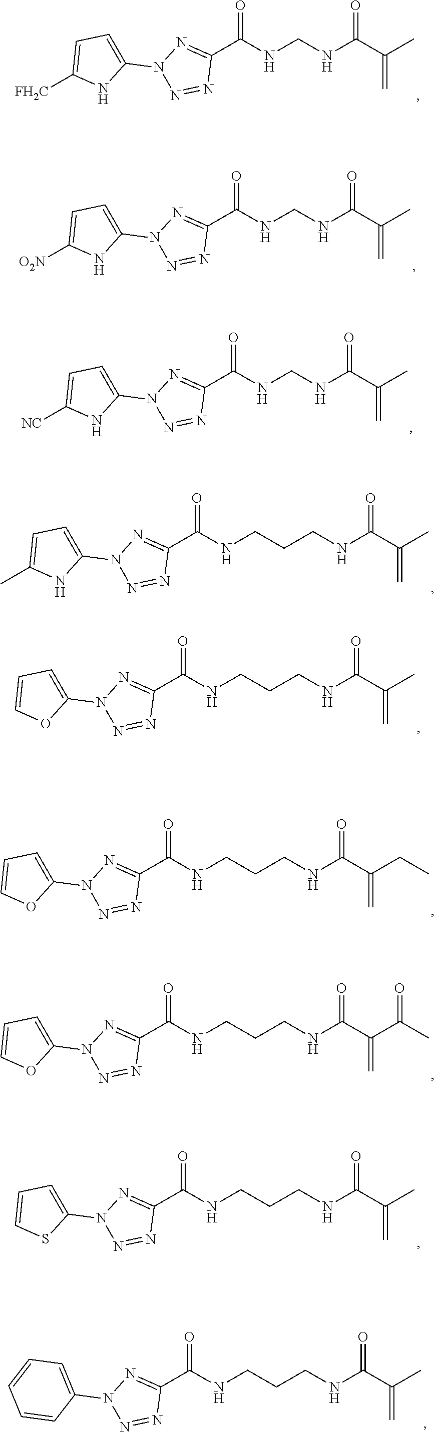

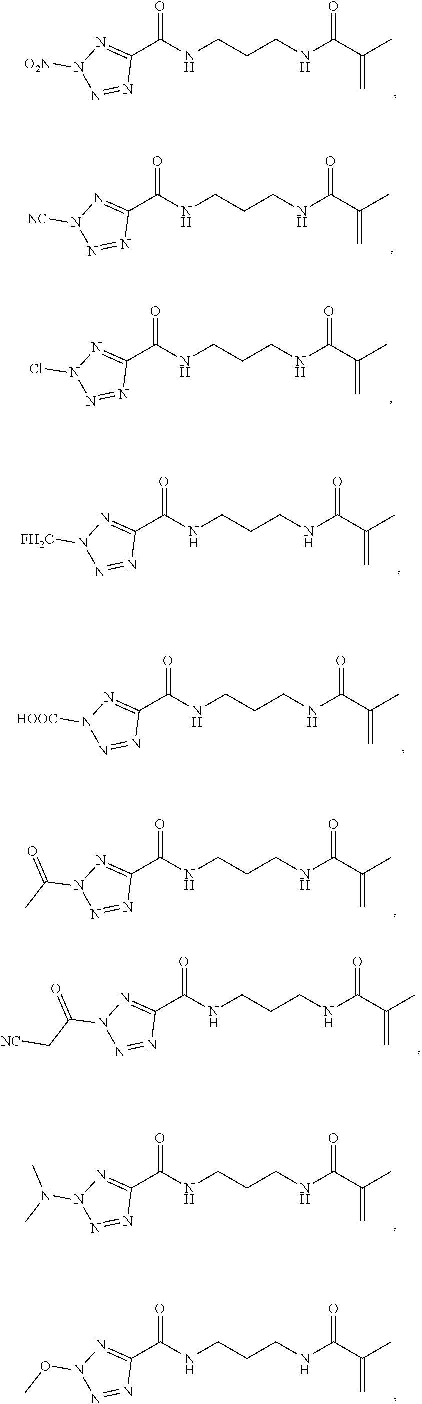

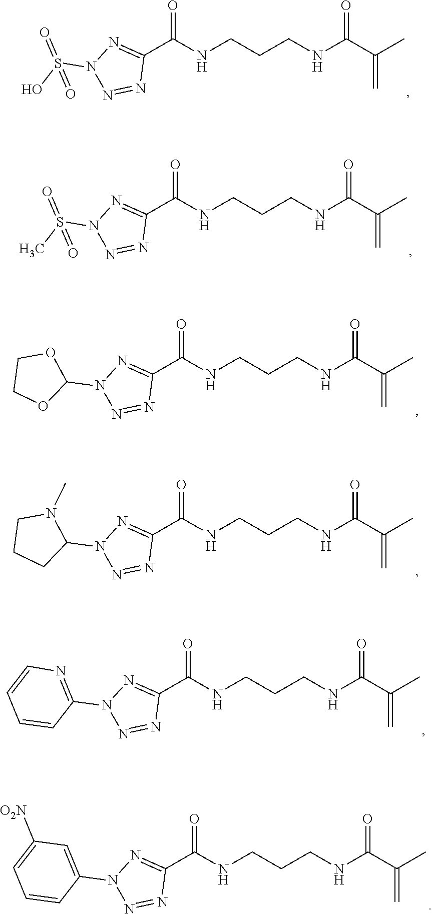

18. A photoactive protein-immobilizing gel, in which the photoactive compound according to claim 1 is contained or immobilized.

19. The photoactive protein-immobilizing gel of claim 18, wherein the gel is a polyacrylamide hydrogel.

20. Use of the photoactive protein-immobilizing gel of claim 18 for separating, immobilizing and/or detecting a protein.
